(19) United States
(12) Patent Application Publication
(10) Pub. No.: US 2008/0243100 A1
(43) Pub. Date: Oct. 2, 2008

(54) Absorbent article having fluid transfer layer

(75) Inventors: Lanying Zhen Wu, Paoli, PA (US); Elizabeth Miller, King of Prussia, PA (US)

Correspondence Address: Covidien, 601 Allendale Road, King of Prussia, PA, 19406, US

(73) Assignee: Tyco Healthcare Retail Services AG, Neuhausen am Rheinfall (CH)

(21) Appl. No.: 12/002,014

(22) Filed: Dec. 14, 2007

Related U.S. Application Data
(60) Provisional application No. 60/920,638, filed on Mar. 29, 2007.

(51) Int. Cl. A61F 13/15

(52) U.S. Cl. 604/385.01

(57) ABSTRACT

A disposable absorbent article having a fluid transfer layer with target insult zone and/or wavy cut edges can have a liquid permeable top sheet, a liquid impermeable back sheet, an absorbent core disposed at least partially therebetween, and a fluid transfer layer disposed between the absorbent core and the top sheet, wherein the target insult zone is provided in the fluid transfer layer and can be defined by a generally centrally located cut-out portion therethrough. At the target insult zone, the fluid passes through the cut-out portion into the absorbent core unimpeded by the fluid transfer layer. The fluid transfer layer and/or the cut-out portion defining the target insult zone can have at least one edge which can be cut in a specially designed pattern, for example, a wavy cut pattern, to provide improved aesthetics.

### CROSS REFERENCE TO RELATED APPLICATION

This application claims priority from U.S. Provisional Application Ser. No. 60/920,638 entitled “Absorbent Article Having Fluid Transfer Layer”, filed on Mar. 29, 2007, the entire content of which is hereby incorporated by reference and relied upon in its entirety.

### BACKGROUND

The absorbent article having a fluid transfer layer with target insult zone and/or wavy cut edges relates generally to disposable absorbent articles, and more particularly to a disposable absorbent article having a target insult zone.

Disposable absorbent articles for adults and children are well known in the art. In the case of adults, as populations continue to increase in longevity, incontinence, a problem of age, presents a need for fluid control in undergarments. In particular, adult incontinence represents a transition from underwear to the use of some type of absorbent article, such as a bladder control pad (“BCP”) to be added to the underwear, or an article known as protective underwear to completely replace it. Other factors can also be associated with adult incontinence, including other unfavorable health problems and physical injury or weakness. All of these things can also result in the affected person being in a physically weakened condition, such that even putting on the disposable absorbent article can be a physically challenging task.

Some examples of BCPs having absorbent cores and associated fluid acquisition layers, or systems, are described in U.S. Pat. No. 7,166,094 (Glaug et al.) entitled “Multiple Layer Absorbent Article” and U.S. Pat. No. 6,838,591 (Waksmundzki et al.) entitled “Absorbent Article With C-Fold Layer Fluid Acquisition System For Use In Absorbent Articles And Method Of Making The Same.”

Another example of such an absorbent article is a feminine hygiene sanitary napkin. In both instances, as I generally true for absorbent articles used by adults, it is desirable for the article to be as discrete as possible, while still being effectively absorbent, so that no one other than the user is cognizant that the wearer is using these types of products.

### SUMMARY

A disposable absorbent article having a target insult zone can generally comprise a liquid permeable top sheet, a liquid impermeable back sheet, an absorbent core disposed at least partially therebetween, and a fluid transfer layer disposed between the absorbent core and the top sheet, wherein the target insult zone is provided in the fluid transfer layer and can be defined by a generally centrally located cut-out portion through the fluid transfer layer. At the target insult zone, the fluid passes through the cut-out portion into the absorbent core unimpeded by the fluid transfer layer. In further embodiments, the fluid transfer layer and/or the cut-out portion forming the target insult zone can have at least one peripheral edge which can be cut in a specially designed pattern to provide improved aesthetics. The specially designed pattern can comprise, for example, a wavy cut pattern. Alternatively, many other types of aesthetically pleasing patterns could also be used.

Further embodiments of the disposable absorbent article can comprise a liquid permeable top sheet, a liquid impermeable back sheet, an absorbent core disposed at least partially therebetween, and a fluid transfer layer disposed between the absorbent core and the top sheet, wherein the fluid transfer layer has at least one peripheral edge cut in a specially designed pattern to provide improved aesthetics. The specially designed pattern can comprise, for example, a wavy cut pattern.

Another embodiment of a disposable absorbent article can comprise a fluid acquisition system having an absorbent core and a fluid transfer layer disposed adjacent a top sheet facing surface of the absorbent core wherein the fluid transfer layer has a cut-out portion through which fluid can pass to the absorbent core unimpeded by the fluid transfer layer. In further embodiments, the fluid transfer layer and/or the target insult zone can have at least one peripheral edge which can be cut in a specially designed pattern to provide improved aesthetics, for example, a wavy cut pattern. Alternatively, many other types of aesthetically pleasing patterns could be used.

Certain illustrative aspects of the disposable absorbent article having the fluid transfer layer with target insult zone and/or wavy cut edges are described herein in connection with the following description and the appended drawing figures. These aspects may be indicative of only a few of the various ways in which the principles of the disposable absorbent article having the fluid transfer layer with target insult zone and/or wavy cut edges may be employed, and which is intended to include all such aspects and any equivalents thereof. Other advantages and features of the disposable absorbent article having the fluid transfer layer with target insult zone and/or wavy cut edges may become apparent from the following detailed description, when considered in conjunction with the appended drawing figures.

### BRIEF DESCRIPTION OF THE DRAWING FIGURES

A more complete understanding of the disposable absorbent article having the fluid transfer layer with target insult zone and/or wavy cut edges can be obtained by considering the following description in conjunction with the accompanying drawing figures, in which:

FIG. 1 is a top plan view of an embodiment of a disposable absorbent article having a target insult zone;

FIG. 2 is a cross section view taken along line II-II in FIG. 1;

FIG. 3 is a top plan view of another embodiment of a disposable absorbent article having a differently shaped target insult zone;

FIG. 4 is a top plan view of a further embodiment of a disposable absorbent article having a differently shaped target insult zone;

FIG. 5 is a top plan view of a further embodiment of a disposable absorbent article having a target insult zone and a fluid acquisition layer with edges cut in a wavy pattern; and

FIG. 6 is a top plan view of an embodiment of a disposable absorbent article having a wavy cut fluid acquisition layer.

### DESCRIPTION OF CERTAIN EMBODIMENTS

The terms “absorbent article” or “article” refer to devices that absorb and contain bodily fluids and other body exudates. More specifically, these terms refer to articles that are placed against or in proximity to the body of a wearer to absorb and contain the various exudates discharged from the body. Disposable absorbent articles are well known in the art. A non-exhaustive list of examples of absorbent articles includes diapers, diaper covers, disposable diapers, training pants, feminine hygiene products and adult incontinence products.

Although a “disposable” absorbent article is described herein, it is to be understood that such “disposable” articles may be intended to be either fully or only partially discarded after a single use. Thus, “disposable” articles can comprise a single inseparable structure, in which the entire article is disposable, or may also comprise articles having replaceable inserts or other interchangeable parts, in which only those inserts or interchangeable parts are disposable.

As used herein, the terms “absorbent article,” “absorbent garment,” or simply “article” or “garment,” refer to devices that absorb and contain bodily fluids and other body exudates. More specifically, these terms refer to articles that are placed against or in proximity to the body of a wearer to absorb and contain the various exudates discharged from the body. Though different in appearance and dimensions, all of the various types of absorbent articles listed previously can generally perform the same basic function of fluid absorption and retention and can all be generally based upon the same fundamental technology. Nearly all of these types of absorbent articles are comprised of a top sheet, a back sheet, and an absorbent core disposed between the top sheet and back sheet. The absorbent core is conventionally located within the article at a position to receive bodily fluids. Optionally, a fluid acquisition layer, or “system,” may also be disposed between the top sheet and the absorbent core.

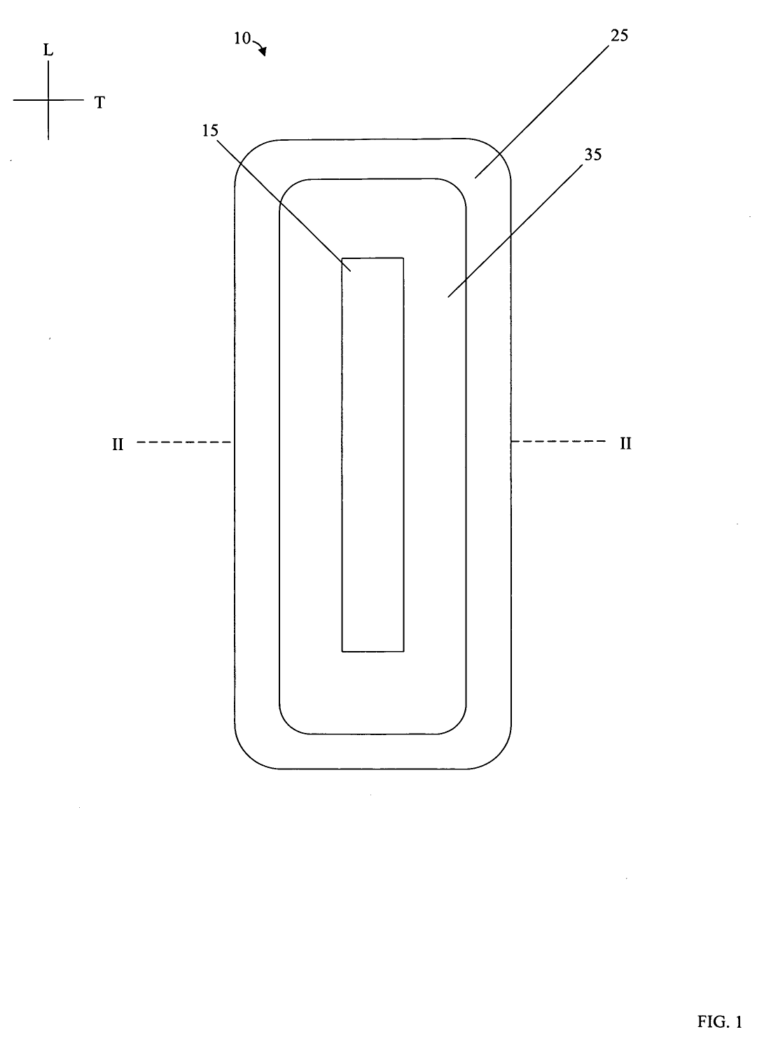

Referring now to the drawing figures, an embodiment of a disposable absorbent article having a target insult zone with the topsheet removed is illustrated in FIGS. 1 and 3-6. The exemplary embodiments shown can comprise a feminine hygiene sanitary napkin, however, this depiction is not intended to limit the various possible embodiments for a disposable absorbent article with a fluid transfer layer having a target insult zone, which can encompass any of the foregoing classes of absorbent articles, without limitation. As used herein, the longitudinal axis L of the article is the dimension of the article corresponding to the front-to-rear dimension of the user, and the transverse axis T is the dimension corresponding to the side-to-side dimension of the user.

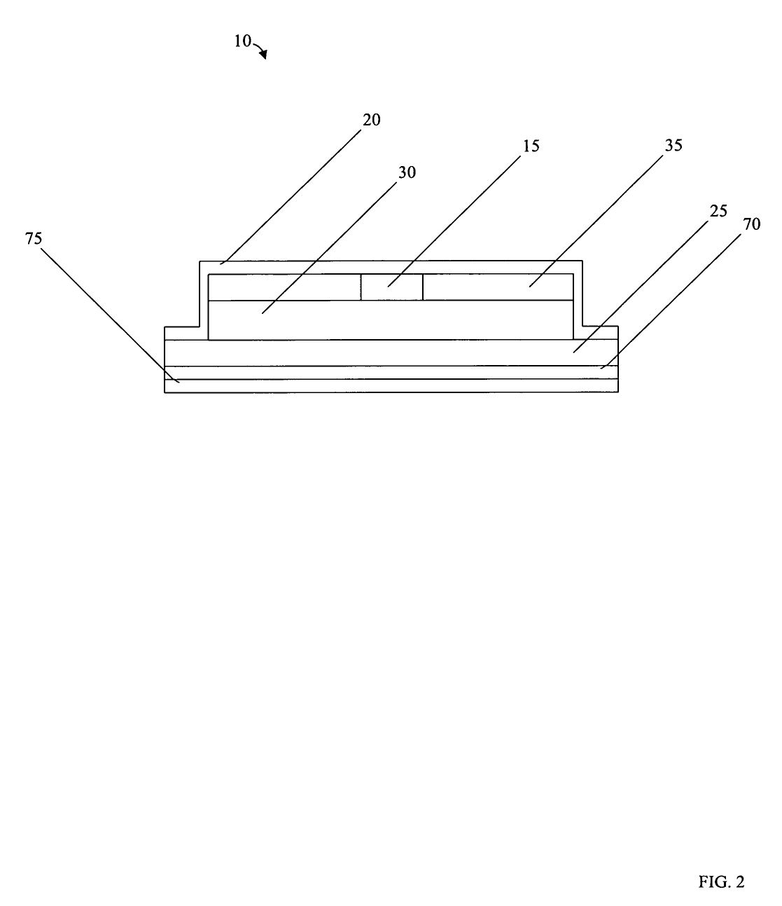

Referring particularly to FIGS. 1 and 2, a disposable absorbent article 10 having a target insult zone 15 can comprise a generally liquid permeable top sheet 20, a generally liquid impermeable back sheet 25, and an absorbent core 30 disposed between at least a portion of the top sheet 20 and at least a portion of the back sheet 25 in a conventional manner. The top sheet 20 can be substantially coterminous with the back sheet 25. When the disposable absorbent article 10 is being worn, the top sheet 20 faces the body of the wearer, whereas the back sheet 25 faces away from the body of the wearer. The article 10 can further comprise a fluid transfer or acquisition layer 35 disposed between the absorbent core 30 and the top sheet 20, wherein the target insult zone 15 is provided in the fluid transfer layer 35, and can be defined by a generally centrally located cut-out 15 through the fluid transfer layer 35. Preferably the topsheet 20 comprises an apertured material which facilitates the flow of exudates through the topsheet to the absorbent core, where fluid is held and helps to prevent flow back to the topsheet, making the absorbent article feel dry to the user.

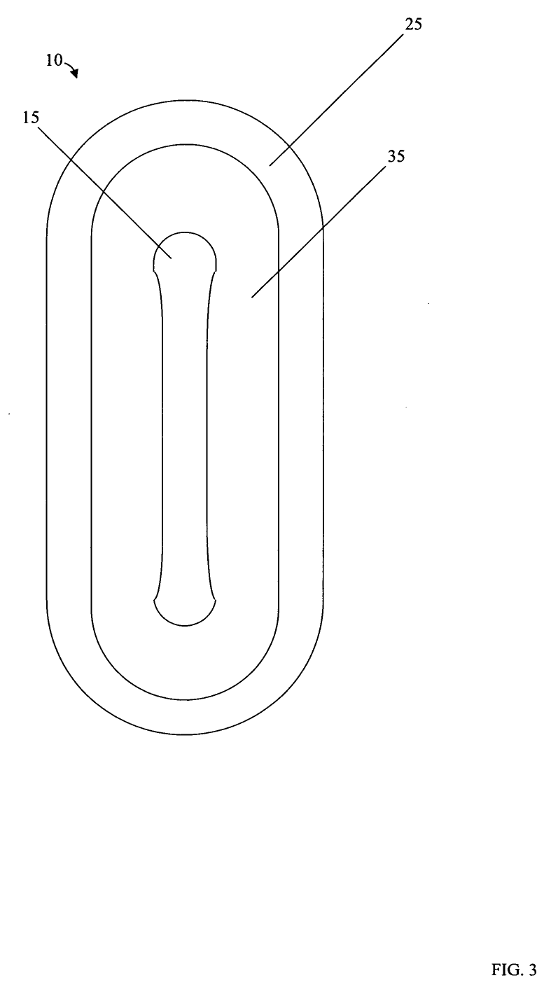

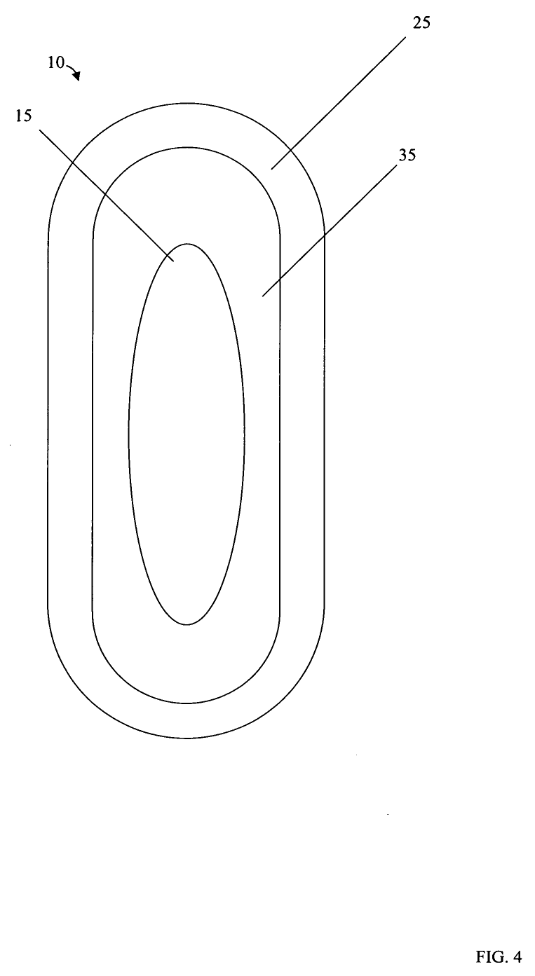

In certain embodiments, the absorbent core 30 can comprise a Dry Formed Composite (“DFC”), pulp material, and Super Absorbent Polymers (“SAP”), or any combination thereof. Also, the absorbent core can be wrapped with white or colored tissue or nonwoven types of material. Another layer of an airlaid or a nonwoven acquisition material 37 can be placed on top of the absorbent core. This layer of material can be a second fluid transfer layer 35, and can be provided with the cut-out 15 which defines the target insult zone 15. The cut-out 15 can be provided in various shapes, such as, for example, dog-bone (FIG. 3), oval (FIG. 4), and various other shapes, including rectangular, diamond, etc.

At the target insult zone 15, the fluid passes through the cut-out 15 into the absorbent core 30 unimpeded by the fluid transfer layer 35. The target insult zone 15 can be provided generally in the center of the fluid transfer layer 35 when it is placed on or over the absorbent core 30. The target insult zone 15 can be made large enough to provide a reservoir for the fluid to pass through the fluid transfer layer 35 unimpeded to the underlying absorbent core 30 and/or additional acquisition layer 37. Additionally, the absorbent core or the acquisition layer 37 can be comprised of colored tissue or nonwoven material, which is visible/exposed through the cut-out 15. This provides a strong visual signal to the wearer as to the position and size of the target insult zone 15. The color of the back sheet 25 and the color of the target insult zone 15 can also be coordinated to further enhance the aesthetics of the absorbent article 10.

A fluid transfer layer 35 is normally used in an absorbent article 10 to improve the fluid handing performance, such as moving the fluid quickly and evenly to the ends of the absorbent core 30 and keeping the rewet low. However, in some cases, a fluid insult can overwhelm the ability of the fluid transfer layer 35 to absorb and redistribute the fluid over the absorbent core 30. If that happens, premature leaking can occur. In order to lessen the likelihood of the fluid transfer layer 35 being overwhelmed by an insult, the aforesaid target insult zone 15 can be provided. By cutting out a portion of the fluid transfer layer 35, thus creating the target insult zone 15, some of the fluid from the insult is immediately passed through to the absorbent core 30 unimpeded by the fluid transfer layer 35. Consequently, it is less likely that the fluid transfer layer 35 will be overwhelmed before the fluid can be redistributed to the underlying absorbent core 30.

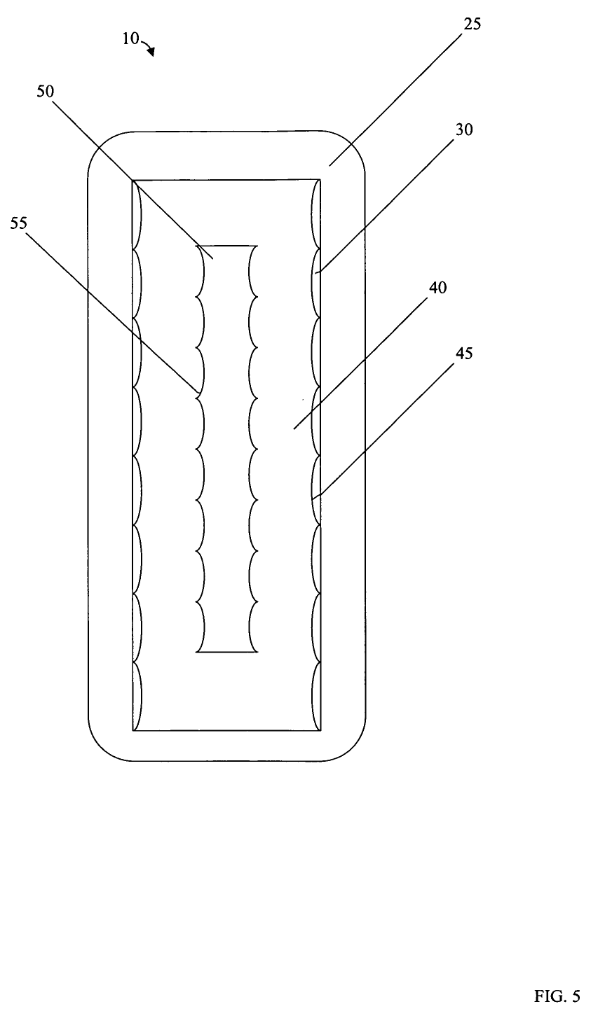

Referring to FIG. 5, in further embodiments, the fluid transfer layer 40 can have at least one peripheral edge which can be cut in a specially designed pattern to provide improved aesthetics. In certain embodiments, both of the longitudinally extending side edges 45 of the fluid transfer layer 40 would be cut in the same specially designed pattern. Alternatively, all edges of the fluid transfer layer 40 may be cut in the specially designed pattern. The specially designed pattern can comprise, for example, a wavy cut pattern. However, many other types of aesthetically pleasing patterns could also be used.

In further embodiments, at least one edge of the generally centrally located cut-out portion 50 which defines the target insult zone 50 can similarly be cut in a specially designed pattern to provide improved aesthetics. In certain embodiments, both of the longitudinally extending side edges 55 of the target insult zone cut-out portion 50 would be cut in the same specially designed pattern, or all edges of the target insult zone 50 may be cut in the specially designed pattern. Similarly, as described above, the specially designed pattern can comprise, for example, a wavy cut pattern. However, many other types of aesthetically pleasing patterns could also be used.

As mentioned previously, a fluid transfer layer 40 is normally used in an absorbent article 10 to improve the fluid handing performance, such as moving the fluid quickly and evenly to the ends of the absorbent core 30 and keeping the rewet low. In order to lessen the likelihood of the fluid transfer layer 40 being overwhelmed by an insult, the aforesaid target insult zone 50 can be provided. The fluid transfer layer 40 material is typically cut into a rectangular shape and placed between the top sheet 20 and the absorbent core 30. Color can be used to make the fluid transfer layer 40 visually noticeable to the consumer.

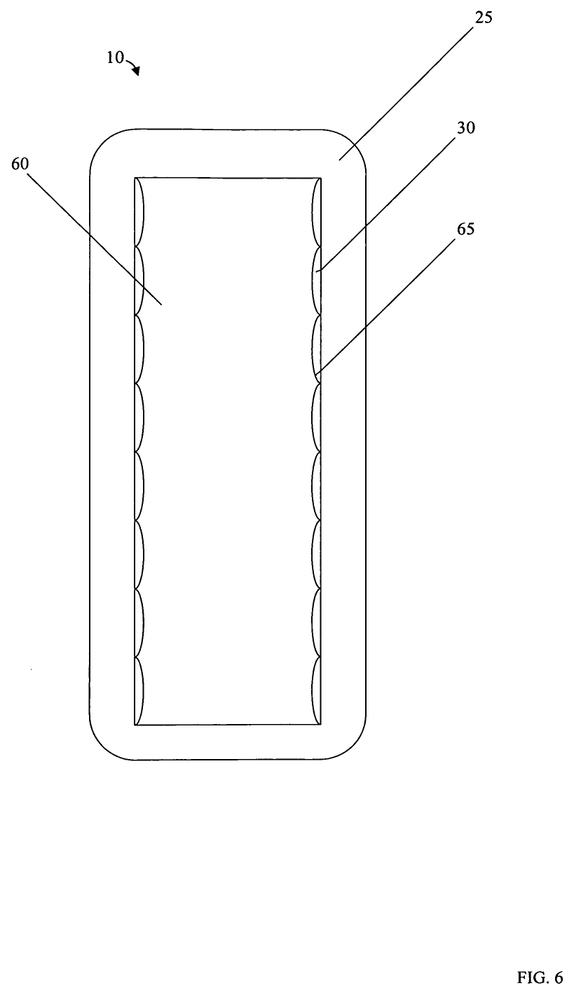

Referring to FIG. 6, further embodiments of a disposable absorbent article 10 can comprise a liquid permeable top sheet 20, a liquid impermeable back sheet 25, an absorbent core 30 at least partially disposed therebetween, and a fluid transfer layer 60 disposed between the absorbent core 30 and the top sheet 20, wherein the fluid transfer layer 60 has at least one peripheral edge cut in a specially designed pattern to provide improved aesthetics. In certain embodiments, both of the longitudinally extending side edges 65 would be cut in the same specially designed pattern. In other embodiments, all edges of the fluid transfer layer 60 may be cut in the specially designed pattern. The specially designed pattern can comprise, for example, a wavy cut pattern. Alternatively, many other types of aesthetically pleasing patterns could be used. The specially designed pattern can improve the aesthetic aspect of the product, thus making the product more pleasing and acceptable to the consumer.

In further embodiments, a fluid acquisition system having a target insult zone can comprise an absorbent core and a fluid transfer layer disposed adjacent a top sheet facing side of the absorbent core wherein the target insult zone is provided in the fluid transfer layer, and can be defined by a generally centrally located cut-out through the fluid transfer layer. As mentioned previously, at the target insult zone, fluid can pass through the cut-out into the absorbent core unimpeded by the fluid transfer layer. Color can be used to make the fluid transfer layer visually noticeable to the consumer. In certain embodiments, at least one peripheral edge of the target insult zone and/or fluid transfer layer can be cut in a specially designed pattern, for example, a wavy cut pattern. Alternatively, many other types of aesthetically pleasing patterns could be used.

Also, as shown in FIG. 2, the absorbent article can include a positioning adhesive 70 and associated release paper 75, as is known in the art.

What has been described above includes exemplary embodiments of a disposable absorbent article having a target insult zone and/or fluid transfer layer having at least one wavy cut peripheral edge. It is, of course, not possible to describe every conceivable combination of components or methodologies for purposes of this description, but one of ordinary skill in the art may recognize that further combinations and permutations are possible in light of the overall teaching of this disclosure. Accordingly, the description provided herein is intended to be illustrative only, and should be considered to embrace any and all alterations, modifications, and/or variations that fall within the spirit and scope of the appended claims. Furthermore, to the extent that the term “includes” may be used in either the detailed description or elsewhere, the use of this term is intended to be inclusive in a manner similar to the term “comprising” as that term is interpreted as a transitional word in a claim.

What is claimed is:

1. A disposable absorbent article comprising:
(a) a top sheet;
(b) a back sheet;
(c) an absorbent core disposed at least partially therebetween;
(d) a fluid transfer layer disposed between said absorbent core and said top sheet; and
(e) wherein said fluid transfer layer has a cut-out portion through which fluid passes to said absorbent core unimpeded by said fluid transfer layer.

2. The disposable absorbent article of claim 1 further comprising said fluid transfer layer having at least one peripheral edge cut in a specially designed pattern to provide improved aesthetics.

3. The disposable absorbent article of claim 2 wherein said specially designed pattern further comprises a wavy cut pattern.

4. The disposable absorbent article of claim 1 further comprising at least one edge of said cut-out portion cut in a specially designed pattern to provide improved aesthetics.

5. The disposable absorbent article of claim 4 wherein said specially designed pattern further comprises a wavy cut pattern.

6. The disposable absorbent article of claim 1 wherein a first color for said back sheet and a second color of at least a portion of said absorbent core visible through said target insult zone are coordinated to provide improved aesthetics.

7. The disposable absorbent article of claim 1, wherein the absorbent article comprises a sanitary napkin.

8. The disposable absorbent article of claim 1, wherein the absorbent article comprises a bladder control pad.

9. A fluid acquisition system for an absorbent article having a topsheet and a back sheet, said fluid acquisition system comprising:
(a) an absorbent core having a top sheet facing side;
(b) a fluid transfer layer disposed adjacent said top sheet facing side of said absorbent core; and
(c) wherein said fluid transfer layer has a cut-out portion through which fluid passes to said absorbent core unimpeded by said fluid transfer layer.

10. The fluid acquisition system of claim 9 wherein said fluid transfer layer has at least one peripheral edge cut in a specially designed pattern to provide improved aesthetics.

11. The fluid acquisition system of claim 10 wherein said specially designed pattern further comprises a wavy cut pattern.

12. The fluid acquisition system of claim 9 further comprising at least one edge of said cut-out portion cut in a specially designed pattern to provide improved aesthetics.

13. The fluid acquisition system of claim 12 wherein said specially designed pattern further comprises a wavy cut pattern.

14. The fluid acquisition system of claim 9 wherein said fluid transfer layer is provided with a first color to make said fluid transfer layer visually noticeable.
